SEQUENCE LISTING (4 sequences)

SEQ ID NO: 1 - protein, 259 aa, Lactococcus lactis
  MISC_FEATURE (1)..(259): EpsA
MQETQEQTIDLRGIFKIIRKRLGLILFSALIVTILGSIYTFFIASPVYTASTQLVVKLPNSDNSAAYAGEVTGNIQMANTINQVIVSPVILDKVRSNLNLSDDSFQKQVTAANQTNSQVIMLTVKYSNPYIAKKIADETAKIFSSDAAKLLNVTNVNILSKAKAQTTPISPKPKLYLAISVIAGLVLGLAIALLKELFDNKINKEEDIEALGLTVLGVTSYDQMSDFNKNTNKNGTQSGTKSSPPSDHEVNRSSKRNKR

SEQ ID NO: 2 - protein, 231 aa, Lactococcus lactis
  MISC_FEATURE (1)..(231): EpsB
MAKNKRSIDNNHYIITSVNPQSPISEQYRTIRTTIDFKMADQGIKSFLVTSSETDEGKTTVSANIAVAFAQQGKKVLLIDGDLRKPTVNITFKVQNRVGLTNILMHQSSIEDAIQGTRLSENLTIITSGPIPPNPSELLASSAMKNLIDSVSDFFDVVLIDIPPLSAVTDAQILSSYVGGVVLVVRAYETKKESLAKTKKKLEQVNANILGVVLHGVDSSDSPSYYYYGVE

SEQ ID NO: 3 - protein, 254 aa, Lactococcus lactis
  MISC_FEATURE (1)..(254): EpsC
MIDIHCHILPGIDDGAKTSGDTLTMLKSAIDEGITTITATPHHNPQFNNESPLILKKVKEVQNIIDEHQLPIEVLPGQEVRIYGDLLKEFSEGKLLKAAGTSSYILIEFPSNHVPAYAKELFYNIKLEGLQPILVHPERNSGIIENPDILFDFIEQGVLSQITASSVTGHFGKKIQKLSFKMIENHLTHFVASDAHNVTSRAFKMKEAFEIIEDSYGSGVSRMLQNNADSVILNESFYQEEPIKIKTKKFLGLF

SEQ ID NO: 4 - protein, 226 aa, Lactococcus lactis
  MISC_FEATURE (1)..(226): EpsD
MEVFEASSELEEPKLVELKKFSRREIIIKRGIDILGGLAGSGLFLIAAALLYVPYKMSSKKDQGPMFYKQKRYGKNGKIFYILKFRTMIINAEQYLELHPEVKAAYHANGNKLESDPRVTKIGSFIRQHSIDELPQFINVLKGDMSLVGPRPILLFEAKEYGERLSYLLICKPGITGYWTTHGRSKVLFPQRADLELYYLQYHSTKNDIKLIMLTIKQILHGSDAY

(19) United States
(12) Patent Application Publication
(10) Pub. No.: US 2008/0219960 A1
(43) Pub. Date: Sep. 11, 2008

(54) Novel Efficient Production Process for Capsular Polysaccharides of Pathogenic Grampositive Bacteria by Heterologous Expression and Secretion of Complex Polysaccharides in Non-Pathogenic, Non-Invasive Gram-Positive Bacteria

(76) Inventors: Masja Nathalie Nierop Groot, Wageningen (NL); Willem Meindert De Vos, Oude Bennekomseweg (NL); Michiel Kleerebezem, Ede (NL)

Correspondence Address: BROWDY AND NEIMARK, P.L.L.C.;624 NINTH STREET, NW, SUITE 300, WASHINGTON, DC, 20001-5303, US

(21) Appl. No.: 11/722,062

(22) PCT Filed: Dec. 16, 2005
(86) PCT No.: PCT/NL2005/050079

§ 371 Date: Dec. 28, 2007

(30) Foreign Application Priority Data
Dec. 16, 2004 (EP) 04078418.3

(51) Int. Cl. A61K 45/00; C12N 1/21; C12N 15/63; A61P 31/00; A61K 31/715; C12P 19/04

(52) U.S. Cl. 424/93.42; 435/252.3; 435/252.31; 435/320.1; 435/101; 424/93.45; 424/93.462; 514/54

(57) ABSTRACT

The current invention provides methods and means for heterologous expression, production and/or secretion of complex capsular polysaccharides in non-pathogenic, non-invasive Gram-positive bacteria. The invention in particular provides non-pathogenic, non-invasive Gram-positive bacteria capable of expression and/or secretion of heterologous, complex polysaccharides from a pathogenic bacterial species. Such bacteria and polysaccharides produced therein may be applied according to the invention to provide compositions for vaccination for the treatment and prevention of infectious bacterial diseases.

### FIELD OF THE INVENTION

The current invention is related to the field of biology, in particular microbiology and heterologous expression of proteins and polysaccharides in bacterial cells. The invention is also related to the field of medicine, in particular the treatment and prevention of infectious diseases, more in particular to the field of vaccination.

### BACKGROUND ART

Microbial polysaccharides (PS's) can be present as capsular polysaccharides (CPSs) covalently associated with the cell-surface, as O-antigens in lipopolysaccharides (LPS) or secreted as extracellular polysaccharides (EPS), and are important virulence factors of both Gram-positive and -negative bacterial pathogens that can cause invasive diseases. Many invasive bacteria produce capsular polysaccharides which are essential virulence factors for pathogen invasion to human body. Capsular polysaccharides are principal antigens found at the cell surface and frequently used for the preparation of vaccines. Vaccination using CPSs, often conjugated to a protein carrier and/or combined with adjuvants stimulating an immune response, is a powerful approach for protecting humankind against infectious diseases caused by bacterial pathogens, such as Streptococcus pneumoniae and Haemophilus influenzae type b (Hib).

Sufficient amounts of safe, pure and well-defined polysaccharides, which are not easily obtained by purification or purely organic synthesis, are crucial for the production of safe and cost effective vaccines. Therefore, heterologous production of capsular polysaccharides in non-pathogenic, non-invasive bacterial host cells is an alternative, efficient, simple, safe and cost-effective solution for purification of CPS from natural sources.

A substantial amount of research on CPSs has been performed on Streptococcus pneumoniae, also referred to as pneumococcus. The pneumococcus is a common cause of infection of the respiratory tract, otitis media and pneumoniae. The most serious forms of pneumococcal disease are pneumonia, meningitis and sepsis. The disease causing ability of S. pneumoniae is clearly associated with its expression of a polysaccharide capsule as all clinical isolates are encapsulated (Whatmore et al., 2000) and spontaneous non-encapsulated variants are avirulent (Velasco et al., 1995). There are currently at least ninety serologically distinct capsule types known for S. pneumoniae (Henrichsen, 1999), each differing in sugar composition en glycosydic linkages (see Weintraub (2003) for an overview).

The treatment of pneumococcal infections has become more complicated and expensive because of the spread of drag-resistant strains of S. pneumoniae. This has urged the need to improve our understanding of the mechanisms involved in regulation of capsule biosynthesis and to develop effective vaccines to prevent pneumococcal infections. Capsular polysaccharides are immunogenic and are used as vaccine antigens. Several vaccines, based on either purified polysaccharides or glycoconjugates thereof, are currently on the market (Weintraub, 2003). The development of effective pneumococcal vaccines is complicated by the antigenic diversity of Streptococcus pneumoniae. The more than 90 pneumococcal serotypes known to date vary greatly in their CPS production and structure. Moreover, the capsular polysaccharides do not reliably induce protective and long lasting immune protection in young children. Current pneumococcal vaccines therefore combine capsular polysaccharides from several serotypes, comprising CPS's from up to 20 serotypes or more. Such multi-serotype vaccines are costly and difficult to produce. It requires culturing of many different pathogenic and hazardous S. pneumoniae serotypes, isolation and extensive purification of CPS, quality control of bacterial strains and the CPS isolates, mixing them in the appropriate or desired quantities and formulation into compositions for vaccination.

Production of pneumococcal CPS of a specific serotype in a pneumococcal cell of another serotype, via homologous recombination of DNA fragments of the cps gene cluster, containing at least some serotype specific cps genes, has also been described (U.S. Pat. No. 5,948,900).

Production of most pneumococcal capsular polysaccharides is expected to occur via a similar pathway as described for O-antigen polysaccharide biosynthesis in Gram-negative bacteria (Whitfield, 1995). The polysaccharide repeat unit is assembled on a lipid carrier, by the sequential action of glycosyltransferase enzymes at the cytoplasmic side of the cell membrane. Once completed, the repeat unit is transported across the cell membrane by the repeat unit transporter and is polymerized at the reducing end of the growing polysaccharide chain (Whitfield and Roberts, 1999) and covalently linked to the cell wall (Sørrensen et al., 1990).

The genetic loci encoding capsule biosynthesis have a cassette-like organization; genes encoding functions required to produce a specific capsule structure are flanked by regulatory genes common to all serotypes. The common region is located upstream of the type-specific genes in the cluster and encodes CpsA, CpsB, CpsC and CpsD (Guidolin et al., 1994; Morona et al., 1997). The exact function of cpsA is still unknown but a function as transcriptional activator was shown for CpsA in Streptococcus agalactiae (Cieslewicz et al., 2001) and mutation of cpsA in S. pneumoniae results in a reduction in capsule amount but did not alter the size distribution of the capsule (Bender et al., 2003). CpsB, CpsC and CpsD were recently shown to be involved in regulation of capsule production via reversible phosphorylation events on tyrosine residues present in CpsD (Bender and Yother, 2001; Bender et al., 2003; Morona et al., 2003). The loci encoding polysaccharide biosynthesis in the non-pathogenic Lactococcus lactis bacterium (Van Kranenburg et al., 1997) but also those present in other non-pathogenic Gram-positive bacteria such as Lactobacillus bulgaricus (Lamothe et al., 2002), Streptococcus thermophilus (Stingele et al., 1996), Lactobacillus plantarum (Kleerebezem et al., 2003), Streptococcus macedonicus (Jolly et al., 2001), Lactobacillus helveticus (Jolly et al., 2002) are organized in a similar way.

The pathogenic and invasive pneumococ and the non-pathogenic, non-invasive lactococci share common features in the machinery for polysaccharide biosynthesis from a CPS or an EPS gene cluster, respectively. It was shown previously that L. lactis is capable of producing the relatively simple pneumococcal type 3 polysaccharide containing a disaccharide repeat unit, upon the introduction of only three type 3 biosynthetic genes including a single processive glycosyltransferase (WO98/31786, Gilbert et al., 2000). Biosynthesis of the simple type 3 polysaccharide involves merely a single glycosyltransferase that forms the glycosidic linkage between a UDP-glucose and UDP-glucuronic acid via a processive mechanism (Cartee et al., 2000). When expressed in L. lactis, pneumococcal serotype 3 CPS remains associated with the L. lactis cell. The cell association hampers a quick separation of CPS from bacterial cells and cell debris. Extensive purification is required, making the purification of CPS from the bacterial cells or bacterial material more difficult and costly.

Examination of the pneumococcal cps loci characterized to date indicates that, except for pneumococcal serotypes 3 and 37, biosynthesis of CPS occurs via the formation of lipid-linked precursor units prior to polymerization of capsular polysaccharides. This is mediated by the action of a so-called priming glycosyl transferase (Kranenburg et al 1999). In contrast to type 3 CPS, production of these more complex capsular polysaccharides could hitherto not be produced in non-pathogenic and non-invasive bacteria, such as for instance Lactococcus sp., by any means. This inability to produce complex pneumococcal CPS in non-pathogenic and non-invasive Gram-positive host bacteria, such as Lactococcus sp., in sufficient quantities, hampers the development of better, safer and cheaper methods for the manufacture of pneumococcal and other vaccines against pathogenic and/or invasive Gram-positive bacteria. Heterologous expression of Gram-positive CPS, such as pneumococcal CPS, in recombinant non-pathogenic and non-invasive Gram-positive bacteria would overcome this problem. Lactic acid bacteria-based production processes have the advantage of being easily up-scalable, robust, low-cost and convenient because no aeration is required.

It is thus an object of the present invention to provide for heterologous expression in non-pathogenic, non-invasive Gram-positive host bacterium of complex CPS from all Gram-positive pathogenic and/or invasive bacteria, such as S. pneumoniae. The invention includes complex forms of CPS which comprise polymerization of repetitive oligosaccharide units that are synthesized via lipid-linked intermediates or lipid-linked precursor units.

### SUMMARY OF THE INVENTION

The present invention provides a Gram-positive bacterium capable of expressing heterologous, complex bacterial CPS. The current invention also pertains to a method for the expression of complex CPS encoding gene clusters in these non-pathogenic and non-invasive Gram-positive host bacteria. The invention discloses suitable recombinant DNA vectors for methods and uses according to the invention. In a further aspect, the invention pertains to a method for the heterologous production and isolation of complex Gram-positive CPS, in particular pneumococcal CPS. In this method CPS is produced by a non-pathogenic, non-invasive Gram-positive host bacterium according to the invention and upon heterologous expression and synthesis using DNA vectors according to the invention, the Gram-positive CPS, in particular pneumococcal CPS, is conveniently secreted into the extracellular space and into the bacterial culture medium, allowing the CPS to he readily and conveniently isolated from the host bacteria or the culture medium. In yet another aspect the invention provides a method for the preparation of pneumococcal CPS compositions, modified forms of CPS and vaccines comprising CPS or modified forms thereof. These compositions will prove particularly useful as vaccines, capable of eliciting in a host an immune response against a pathogenic, invasive Gram-positive bacterium. Merely as a non limiting example, the invention is illustrated by expression of the complex pneumococcal type 14 CPS in Lactococcus lactis, using the DNA vectors and methods of the current invention.

### DETAILED DESCRIPTION OF THE INVENTION

### Definitions

A gene cluster is a stretch of DNA comprising a set of closely related genes that code for the same or similar proteins and which are usually grouped together on the same chromosome or plasmid and expression and translation of which is regulated for the cluster as a whole. In bacteria a gene cluster is also referred to as an operon: a functional unit consisting of a promoter, an operator and a number of structural genes, found mainly in prokaryotes. The structural genes commonly code for several functionally related enzymes, and although they are transcribed as one (polycistronic) mRNA each is independently translated. In the typical operon, the operator region acts as a controlling element in switching on or off the synthesis of mRNA. A genetic unit consisting of a feedback system under the control of an operator gene, in which a structural gene transcribes its message in the form of mRNA upon blockade of a repressor produced by a regulator gene. Included here is often an attenuator site of bacterial operons where transcription termination is regulated.

A DNA vector as defined in this application may be any DNA vector known in the art of molecular cloning; any virus, phage, phagemid, cosmid, BAC, episome or plasmid.

Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps). Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide may be operably linked to another promoter sequence or, if applicable, another secretory signal sequence and/or terminator sequence than in its natural environment.

When used to indicate the relatedness of to nucleic acid sequences the term “homologous” means that one single-stranded nucleic acid sequence may hybridise to a complementary single-stranded nucleic acid sequence. The degree of hybridisation may depend on a number of factors including the amount of identity between the sequences and the hybridisation conditions such as temperature and salt concentration as generally known to the skilled person. Preferably the region of identity is greater than about 5 bp, more preferably the region of identity is greater than 10 bp.

As used herein, the term “promoter” refers to a nucleic acid fragment that functions to control the transcription of one or more genes, located upstream with respect to the direction of transcription of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most environmental and physiological conditions. An “inducible” promoter is a promoter that is active only under specific environmental or physiological conditions.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

Vaccine: A substance or group of substances meant to cause the immune system to respond to a tumor or to microorganisms, such as bacteria or viruses. A vaccine can help the immune system of a subject to recognize and combat infections and to destroy cancer cells or microorganisms and or virally infected cells. A vaccine (named after vaccinia, the infectious agent of cowpox, which, when innoculated, provides protection against smallpox) is used to prepare a human or animal's immune system to defend the body against a specific pathogen, usually a bacterium, a virus or a toxin. Depending on the infectious agent to prepare against, the vaccine can be a weakened bacterium or virus that lost its virulence, or a toxoid (a modified, weakened toxin or particle from the infectious agent). The immune system recognizes the vaccine particles as foreign, destroys them and “remembers” them. When the virulent version of the agent comes along, the immune system is prepared for a fast strike, neutralizing the agent before it can spread and multiply to vast numbers. Live but weakened (attenuated) vaccines are used against tuberculosis, rabies, and smallpox; killed agents are used against cholera and typhoid; toxoids against diphtheria and tetanus.

### Embodiments of the Invention

In a first embodiment, the current invention provides a non-pathogenic, non-invasive Gram-positive bacterium that comprises;

a) a first heterologous DNA fragment comprising capsular polysaccharide (CPS) serotype specific genes of a Gram-positive bacterial species,

b) a second DNA fragment comprising the common, regulatory genes and a priming-glycosyltransferase obtained from a Gram-positive bacterium different from the bacterium under a),

c) and upon expression of said fragments produces heterologous polysaccharides of the bacterial species under a). In particular the invention provides non-pathogenic and/or non-invasive Gram-positive bacteria expressing heterologous serotype specific cps genes of pathogenic and/or invasive Gram-positive bacterial species producing complex type CPS. Complex CPS as defined in this specification comprises CPS which is produced in vivo as a polymer of repetitive oligosaccharide units that are synthesized via lipid-linked intermediates. In more detail, complex capsular polysaccharides comprise a polymer of repetitive multicomponent units of at least four sugars. Repetitive units are assembled intracellularly on lipid carriers by the sequential action of glycosyltransferases that link a monosaccharide unit to the lipid linked intermediate. Once complete, lipid linked repeat units are transported across the cell membrane and polymerized by a polymerase enzyme. In contrast, CPS of the simple type, for instance CPS of Streptococcus pneumoniae serotypes 3 and 37 synthesis involves a single glycosyltransferase (Arrecubieta et al., 1996; Llull et al., 2001) that directly transfers monosaccharides to the growing polysaccharide chain (Cartee et al., 2000) without the intervention of a lipid-linked intermediate. Additionally, this glycolsyltransferase appears to transport the growing polysaccharide chain across the membrane.

In a preferred embodiment the invention provides Gram-positive bacteria which secrete (at least part of) the complex polysaccharides into the extracellular space, more preferably into the culture medium. A fraction of the CPS may thus be retained in the cell envelope of the host cell, however, preferably a major fraction of the CPS is secreted into the culture medium, which is particularly advantageous for (continuous) production or purification purposes.

Preferably the bacterial host expressing heterologous, complex CPS is selected from the group of non-pathogenic and/or non-invasive, Gram-positive bacteria consisting of Lactobacillus, Lactococcus, Pediococcus, Carnobacterium, Bifidobacterium, Oenococcus, Bacillus subtilis, Streptococcus thermophilus, and other non-pathogenic and/or non-invasive Gram-positive bacteria known in the art. The bacterial host cell preferably is a Gram-positive bacterium, more preferably a Gram-positive bacterium that belongs to a genus selected from the group consisting of Lactobacillus, Lactococcus, Leuconostoc, Carnobacterium, Bifidobacterium, Bacillus, Streptococcus, Propionibacterium, Oenococcus, Pediococcus, Enterococcus. Most preferably the bacterial host cell is a bacterium that belongs to a species selected from the group consisting of L. acidophilus, L. amylovorus, L. bavaricus, L. brevis, L, caseii, L. crispatus, L. curvatus, L. delbrueckii, L. delbrueckii subsp. bulgaricus, L. fermentum, L. gallinarum, L. gasseri, L. helveticus, L. jensenii, L. johnsonii, L. minutis, L. murinus L. paracasei, L. plantarum, L. pontis, L. reuteri, L. sacei, L. salivarius, L. sanfrancisco, Lactobacillus ssp., C. piscicola, B. subtilis, Leuconostoc mesenteroides, Leuconoctoc lactis, Leuconostoc ssp, L. lactis subsp. lactis, L. lactis subsp. cremoris, Streptococcus thermophilus, B. bifidum, B. longum, B. infantis, B. breve, B. adolescente, B. animalis, B. gallinarum, B. magnum, B. thermophilus.

The serotype specific sequences are preferably obtained from Gram-positive bacteria producing complex type CPS as herein defined. Preferably the type specific genes are obtained from the group of pathogenic and/or invasive Gram-positive CPS producing bacteria within the genera Streptococcus, Staphylococcus, Enterococcus, Bacillus, Listeria, Corynebacterium, Clostridium, or may be Gram-positive pathogens, such as Streptococcus sp. relevant to the veterinary field (e.g, livestock, pet animals). More in particular, the serotype specific genes are obtained from well known Gram-positive pathogens such as, but not limited to, Streptococcus pneumoniae, Enterococcus faecalis, Streptococcus mutans, Streptococcus pyogenes, Staphylococcus aureus, Streptococcus agalactiae, Streptococcus epidermidis, Streptococcus gordonii, Streptococcus mitis, Streptococcus oralis, Streptococcus equi, Bacillus anthracis and Staphylococcus aureus.

In a most preferred embodiment of the invention and as illustrated in the examples section, the invention provides non-pathogenic and/or non-invasive Gram-positive bacteria producing complex CPS from heterologous serotype specific genes obtained from Streptococcus pneumoniae serotypes producing complex capsular polysaccharides. These serotypes comprise all Streptococcus pneumoniae serotypes known to date, with the exception of serotypes 3, 37 and potentially other pneumococcal serotypes which produce a different type of CPS having a more simple structure and for which biosynthesis does not require linkage of lipid linked precursor units. The use of type specific genes from pneumococcal serotypes (Danish nomenclature) 1, 2, 4, 5, 6A, 6B, 7A, 7B, 7C, 7F, 8, 9A, 9L, 9N, 9V, 10A, 10B, 10C, 11A, 11B, 11C, 11F, 12B, 12F, 13, 14, 15F, 15A, 15B, 15C, 16F, 16A, 17F, 17A, 18A, 18B, 18C, 19F, 19A, 19B, 19C, 20, 21, 22F, 22A, 23A, 23B, 24F, 24A, 24B, 25F, 25A, 27, 28F, 28A, 29, 33F, 33A, 33B, 33C, 10F, 11D, 12A, 18F, 23F, 31, 32F, 32A, 33D, 34, 35F, 35A, 35B, 35C, 36, 38, 39, 40, 41F, 41A, 42, 43, 44, 45, 46, 47F, 47A, 48 are particularly preferred embodiments of the invention.

The non-pathogenic and/or non-invasive Gram-positive host bacterium according to the invention comprises and expresses common, regulatory EPS or CPS genes obtained from an EPS or CPS gene cluster of a Gram-positive bacterium. Preferably said common, regulatory EPS or CPS genes comprise at least the common, regulatory genes epsA (or cpsC), epsB (or cpsD), and optionally epsC (or cpsB). Preferably the common, regulatory EPS or CPS genes also comprise a priming glycosyltransferase (GTF) of a Gram-positive EPS or CPS gene cluster, encoded by the cpsD or cpsE genes.

There are several characteristic features for EpsA, EpsB, EpsC and EpsD homo logs. EpsC homologs generally contain conserved PHP (polymerase and histidinol phosphatase) motifs (Aravind and Koonin, 1998). EpsA homologs contain two conserved transmembrane segments and EpsB contains conserved nucleotide-binding motifs (Fath and Kolter, 1993). Phospho-glycosyltransferases like EpsD can be recognized by the presence of conserved A, B, and C blocks described previously by (Wang et al., 1996; Van Kranenburg et al. 1999). This glycosyltransferase (GTF) links the first sugar to a lipid carrier (most likely undecaprenylphosphate) and is conserved among Gram-positives (at least 30% identity) and is therefore designated the priming glycosyltransferase.

Preferably the epsA gene encoded protein that is expressed by a Gram-positive host cell according to the invention, shares at least 20, 30, 40, 50, 60, 70, 80 or 90% amino acid identity with Lactococcus lactis EpsA, the epsB or epsC encoded protein shares at least 20, 30, 40, 50, 60, 70, 80 or 90% amino acid identity with Lactococcus lactis EpsB and the epsD encoded protein shares at least 30, 40, 50, 60, 70, 80 or 90% amino acid identity with Lactococcus lactis EpsD. Amino acid sequences of L. lactis EpsA, EpsB, EpsC and EpsD are provided in the sequence listing, SEQ ID No's 1 to 4 respectively and are published by Van Kranenburg et al. (1997). Swiss-Prot database numbers are O06029, O06030, O06031, O06032 for EpsA, EpsB, EpsC and EpsD, respectively.

In another aspect, the invention provides a DNA vector capable of conferring heterologous expression of Gram-positive complex CPS serotype specific genes in a non-pathogenic and/or non-invasive Gram-positive bacterial host cell. The DNA vector comprises a DNA fragment encoding Gram-positive complex CPS serotype specific cps genes, wherein one or more of the cps genes are selected from the group consisting of type specific genes obtained from a capsular polysaccharide gene cluster.

Said serotype-specific genes are located within a 20 kb region immediately downstream of the common, regulatory genes in the polysaccharide biosynthesis gene clusters. The serotype-specific regions generally encode glycosyltransferases, a highly hydrophobic polymerase with 9-14 predicted transmembrane segments and a protein involved in the transport of repeat units mat generally contains 12-14 membrane-spanning domains. The glycosyltransferases encoded by the type-specific region can be nucleotide diphospho-sugar, nucleotide monophospho-sugars and sugar phosphates (EC 2.4.1.x) and can be classified in distinct sequence-based families as first described by Campbell et al., (1997).

Optionally and preferably the serotype-specific fragment of DNA sequences comprising the serotype specific cps genes does not contain the priming glycosyltransferase encoding gene. The vector according to the invention may or may not comprise or provide expression for the common regulatory eps/cps genes. Preferably, the DNA vector according to the current invention comprising Gram-positive serotype specific cps genes, does not comprise functional common regulatory eps/cps genes and does not provide gene expression for: epsA/cpsC, epsB/cpsD, epsC/cpsB and/or epsD/cpsE.

The type specific genes expressed in a vector according to the invention may be selected from the group consisting of cpsE, cpsF, cpsG, cpsH, cpsI, cpsJ, cpsK, cpsL, or homo logs thereof. Preferably all type specific genes of a CPS producing gene cluster, or all type specific genes expression of which is essential for CPS production, are cloned in and expressed from a DNA vector according to the invention. The vector may be any DNA vector known in the art such as a phage or virus, phagemid, cosmid or BAC (bacterial artificial chromosome) vector, and preferably is a plasmid. Also vectors suitable for homologous recombination, gene replacement and/or genomic integration are encompassed within the scope of the invention. The type specific genes may also be expressed from several different vectors within one host cell, for instance to overcome cloning size restrictions of a chosen DNA vector. The choice of vector elements, such as for example the choice of the transcription regulatory sequence, vector backbone, selectable marker encoding sequences, origin of replication, enhancer elements, etc., depends on the host cell in which transcription and translation are to be achieved and is easily determined by the skilled person. In principle, any transcription regulatory element that is active in the host cell may be used, and it may be homologous or heterologous to the host cell. For efficient transcription in prokaryotic cells, such as gram-positive bacteria, preferably prokaryotic transcription regulatory sequences should be used, while for transcription and translation in eukaryotic host cells preferably elements of eukaryotic origin are used. In one embodiment preferably a promoter which is homologous to the host cell is used. Strong constitutive promoters and promoters which are strongly induced following induction are especially preferred.

In a highly preferred embodiment of the invention, the type specific cps genes are obtained from the group of CPS producing Streptococcus pneumoniae serotypes, that produce complex CPS that is synthesized as previously defined herein; by polymerization of repetitive, lipid linked precursor oligosaccharide units. Such type specific genes may be obtained from the pneumococcal serotypes (Danish nomenclature) 1, 2, 4, 5, 6A, 6B, 7A, 7B, 7C, 7F, 8, 9A, 9L, 9N, 9V, 10A, 10B, 10C, 11A, 11B, 11C, 11F, 12B, 12F, 13, 14, 15F, 15A, 15B, 15C, 16F, 16A, 17F, 17A, 18A, 18B, 18C, 19F, 19A, 19B, 19C, 20, 21, 22F, 22A, 23A, 23B, 24F, 24A, 24B, 25F, 25A 27, 28F, 28A, 29, 33F, 33A, 33B, 33C, 10F, 11D, 12A, 18F, 23F, 31, 32F, 32A, 33D, 34, 35F, 35A, 35B, 35C, 36, 38, 39, 40, 41F, 41A, 42, 43, 44, 45, 46, 47F, 47A, 48, and are particularly preferred.

In a preferred embodiment of the invention, serotype specific cps genes in a DNA vector are under transcriptional control of EPS or CPS gene cluster regulatory sequences. Said EPS or CPS gene cluster regulatory sequences may be from a gene cluster different than the gene cluster from which the serotype specific genes were obtained, and are preferably EPS or CPS gene cluster regulatory sequences from the non-pathogenic and/or non-invasive Gram-positive bacterial host cell that is used and/or the gene cluster from which the common regulatory eps or cps genes were obtained.

In another preferred embodiment the serotype specific cps genes are comprised within a polycistronic transcriptional unit under control of a Gram-positive EPS or CPS gene cluster regulatory sequences, optionally replacing or partly replacing the serotype specific cps or eps genes in the cluster. However, apart from EPS or CPS gene cluster regulatory sequences, also other bacterial, viral, artificial or even mammalian regulatory sequences such as promoters, enhancers, attenuators, insulators, terminators known in the art may be advantageously applied. Cloning and bacterial transformation methods, DNA vectors and the use of regulatory sequences are well known to the skilled artisan and may for instance be found in Current Protocols in Molecular Biology, F. M. Ausubel et al, Wiley Interscience, 2004, incorporated herein by reference.

The DNA vectors comprising Gram-positive serotype specific genes according to the current invention are preferably transferred, by means known in the art per se, (for instance transformation or transduction) to a non-pathogenic, non-invasive Gram-positive host bacterium of different species or serotype than the serotype specific cps genes in the DNA vector.

In another aspect the invention provides a method for the heterologous production of complex capsular polysaccharides (CPS) in a non-pathogenic, non-invasive Gram-positive bacterium, comprising the steps of;

a) culturing the bacterium according to the current invention, comprising a vector and/or DNA fragment according to the invention, under conditions conducive of CPS production,
b) recovery of the produced complex CPS from the bacterial cells.
In a preferred embodiment the CPS produced by the non-pathogenic and/or non-invasive Gram-positive bacterial host cell is secreted into the extracellular environment and is not, or only partially retained in the cell envelope, allowing the extracellular polysaccharides produced to be recovered from the bacterial culture medium. Methods for the purification of EPS/CPS are known in the art and may for instance be found in Looijesteijn and Hugenholtz (1999) or Gonçalves et al. (2003).

In yet another embodiment the invention provides pharmaceutical compositions comprising capsular polysaccharides, preferably capsular polysaccharides from pathogenic and/or invasive Gram-positive bacteria, that have been produced by and obtained from non-pathogenic and/or invasive Gram-positive bacterial host cells according to the current invention.

In a first embodiment a pharmaceutical or nutraceutical composition according to the invention may comprise the non-pathogenic and/or non-invasive bacterial cells according to the invention in a viable, for instance life attenuated, or non-viable forms, for instance by heat treatment of formalin treatment. The pharmaceutically acceptable composition comprising the bacterium may comprise one or more excipients or immunogenic adjuvants known in the art. Pharmaceutically acceptable adjuvants are known to the skilled artisan and may be found in textbooks such as Remmington's Pharmaceutical Sciences, 18 th ed. Mack Publishing Company, 1990 and Current Protocols in Immunology, Edited by: John E. Coligan. Wiley Interscience, 2004.

In another embodiment, the pharmaceutically acceptable composition according to the current invention may comprise isolated and/or purified complex polysaccharides, preferably capsular polysaccharides of a pathogenic and/or invasive Gram-positive bacterium such as pneumococcal CPS, that is produced in, arid obtained from a non-pathogenic, non-invasive Gram-positive bacterial host cell as herein described. The pharmaceutical composition according to the invention may further comprise one or more excipients and/or immunogenic adjuvants known in the art of vaccination or immunisation.

Preferably, the pharmaceutical compositions according to the invention are compositions suitable to be applied as compositions for vaccination or immunisation purposes. In one embodiment, in such a composition or vaccine, the CPS molecules are covalently attached to an immunological molecule, such as e.g. an antigenic protein, including e.g. a tetanus toxoid, diphtheria toxoid, meningococcal outer membrane proteins, diphtheria protein CRM₁₉₇ and other immunogenic molecules known in the art. Immunogenic proteins and adjuvant molecules which may be suitably applied in the compositions according to the invention are polyIC, LPS, Lipid A, Poly-A-poly-U, GERBU®, RIBI®, Pam3®, Specol®, Freunds, Titermax® and other adjuvants known and used in the art.

The compositions and vaccines comprising Gram-positive CPS obtained from a non-pathogenic and/or non-invasive Gram-positive bacterium according to the invention may be administered orally or intranasally or intraveneously according to methods known in the art of vaccination. Pneumococcal vaccines according to the current invention may for instance be administered as described in U.S. Pat. No. 6,224,880 and references therein.

### FIGURE LEGENDS

FIG. 1

Schematic representation of the plasmids used for polysaccharide production in L. lactis. Panel A: B40 eps gene cluster of plasmid pNZ4030; Peps, promoter of the eps gene cluster. Panel B: an in-frame deletion of epsABCD from pNZ4030, excission of the resulting gene cluster by NcoI digestion and ligation into NcoI-digested pIL253 results in pNZ4220. Panel C: excission of the epsEFGHIJKL or fY genes by BamHI digestion and replacement with a 6.8 kb fragment encompassing cps14FGHIJKL results in pNZ4230. The HindIII restriction site used for cloning of the PCR-amplified cps14 genes (described in Materials and Methods) is indicated. Panel D: Plasmids containing the B40 eps (pNZ4206) and the cps14 (pNZ4237) regulatory genes and under control of the nisin-inducible promoter and several derivative constructs used in this study (see Materials and Methods section for more details).

FIG. 2

Immunodetection of type 14 PS produced in L. lactis strains. Ten μl of culture supernatant of either induced or non-induced cells and 10 μl of induced cell suspension were spotted onto nitrocellulose membranes and detected with type 14-specific antiserum. L. lactis harbouring either pNZ4220 and pNZ4206 (lane 1); pNZ4230 (lane 2); pNZ4230+pNZ4206 (lane 3); pNZ4230+pNZ4209 (lane 4); pNZ4230+pNZ4208 (lane 5); pNZ4230+pNZ4235 (lane 6); pNZ4230+pNZ4237 (lane 7); pNZ4230+pNZ4238 (lane 8); pNZ4230+pNZ4221 (lane 9).

FIG. 3

Panel A. NMR spectrum of polysaccharide purified from S. pneumoniae serotype 14 Panel B. NMR spectrum of polysaccharide purified from L. lactis expressing pNZ4230 and pNZ4206

FIG. 4

Tyrosine phosphorylation of EpsB or Cps14D proteins in B40 polysaccharide and pneumococcal type 14 polysaccharide producing L. lactis strains.

Panel A. Cell extracts of L. lactis harbouring pNZ4230 in combination with pNZ4206 (lane 1), pNZ4209 (lane 2), pNZ4208 (lane 3), pNZ4237 (lane 4, 5), pNZ4235 (Lane 6, 7), pNZ4238 (lane 8), pNZ4221 (lane 9). Cells were either induced (lane 1, 2, 3, 4, 6, 8, 9) with 1 ng/ml nisin or uninduced (lane 5, 7).

Panel B. Cell extracts of L. lactis harbouring pNZ4220 in combination with pNZ4206 (lane 1), pNZ4209 (lane 2), pNZ4208 (lane 3), pNZ4237 (lane 4, 5), pNZ4235 (Lane 6, 7), pNZ4238 (lane 8), pNZ4221 (lane 9). Cells were either induced (lane 1, 2, 3, 4, 6, 8, 9) with 1 ng/ml nisin or uninduced (lane 5, 7).

Tyrosine-phosphorylated protein was detected by using Western immunoblotting with a mouse monoclonal antibody against phosphotyrosine.

### EXAMPLES

### Example 1

### Streptococcus pneumoniae Serotype 14 CPS Synthesis and Secretion in L. lactis

### Materials and Methods

### Bacterial Strains and Growth Conditions

All bacterial strains and plasmids used in this study are listed in Table 1. L. lactis was grown at 30° C. without aeration in M17 broth (Merck, Darmstadt, Germany), supplemented with 0.5% (wt/vol) glucose or in chemically defined medium (Looijesteijn and Hugenholtz, 1999) supplemented with 2% (wt/vol) glucose. Escherichia coli, which was used as cloning host, was grown with aeration in Trypton-Yeast (TY) broth (Sambrook et al., 1989) at 37° C. Where appropriate, media were supplemented with erythromycin (5 μg/ml), chloramphenicol (5 μg/ml), or tetracycline (2.5 μg/ml).

TABLE 1
Strains and plasmids used

| | | |
|---|---|---|
| Strain or plasmid | Relevant properties | Reference |

Strains

| | | |
|---|---|---|
| L. lactis | | |
| NZ9000 | MG1363 pepN::nisRK | Kuipers et al., 1998 |
| E. coli E10 | | |

Plasmids

| | | |
|---|---|---|
| pNZ4130 | Tet^{R}, pNZ4000 derivative harbouring B40 eps | Boels et al., 2001 |
| | gene cluster | |
| pNZ84 | Cm^{R}, pACYC184 derivative, cloning vector for | Van Alen-Boerrigter et al., 1991 |
| | E. coli | |
| pIL253_Ncol | Ery^{R}, lactococcal cloning and expression | Boels et al., 2003 |
| | vector | |
| pNZ124 | Cm^{R}, lactococcal cloning vector | Platteeuw et al., 1993 |
| pUC18ery | Amp^{R}, Ery^{R} | Van Kranenburg et al., 1997 |
| pNZ8148 | Cm^{R}, lactococcal cloning and expression | Reference |
| | vector | |
| pNZB020 | Cm^{R} lactococcal cloning and expression vector | De Ruyter et al., 1996 |
| pNZ4222 | Ery^{R}, vector for epsABCD deletion in B40 eps | This study |
| | gene cluster | |
| pNZ4200 | PNZ4130 derivative carrying an in-frame | This study |
| | deletion of epsABCD | |
| pNZ4220 | pIL253_Ncol derivative carrying the | This study |
| | ΔepsABCD eps gene cluster from pNZ4200 | |
| pNZ4206 | pNZB148 derivative carrying epsABCD | Nierop Groot et al., 2004 |
| pNZ4231 | pNZ8020 derivative carrying cps14B | This study |
| pNZ4209 | pNZ4206 derivative harbouring EpsB_{ΔYYYY}, | Nierop Groot et al., 2004 |
| pNZ4208 | pNZ4206ΔepsC | Nierop Groot et al., 2004 |
| pNZ4090 | Cm^{R}, pNZ8020 derivative carrying cps14′EF′ | Van Kranenburg et al., 1999 |
| pNZ4233 | Cm^{R}, pNZ8020 derivative harbouring cps14CD | This study |
| pNZ4235 | pNZ4233 derivative containing cp14CDE | This study |
| pNZ4237 | pNZ4235 derivative containing cp14BCDE | This study |
| pNZ4221 | pNZ8148 derivative containing epsABC and | This study |
| | cps14E | |
| pNZ4238 | pNZ4237 derivative containing a 5′-end | This study |
| | truncated cps14E | |
| pMK104 | pBlueScript II KS derivative harbouring | Kolkman et al., 1997 |
| | cps14CD | |
| pNZ84cpsF-J | Cm^{R}, pNZ84 derivative harboring cps14FGHIJ′ | This study |
| pNZ84cpsJ-L | Cm^{R}, pNZ84 derivative harbouring cps14J′KL | This study |
| pNZ84cpsF-L | Cm^{R}, pNZ84 derivative harbouring | This study |
| | cps14FGHIJKL | |
| pNZ4230 | Ery^{R}, pNZ4220 derivative harbouring the | This study |
| | cps14FGHIJKL genes under control of the B40 | |
| | eps promoter | |

### DNA Manipulations and Sequence Analysis

Small-scale isolation of E. coli plasmid DNA and standard recombinant DNA techniques were performed as described by Sambrook et al., (1989).

For large scale plasmid isolations of E. coli, JetStar columns (Genomed GmbH, Bad Oberhausen, Germany) were used following the instructions of the manufacturer. Isolation and transformation of L. lactis plasmid DNA was performed as described previously (de Vos et al., 1989).

### Construction of Plasmids

A derivative of the B40 eps plasmid pNZ4130 was constructed that contains an in-frame deletion of the epsABCD genes. Using primer combinations EPSRF1/EPSAR1 and EPSDF2/EPSFR2, pNZ4030 as the template and Pwo polymerase (Roche Diagnostics GmbH, Mannheim, Germany) 1 kb amplicons were obtained by PCR. The two obtained PCR products were digested XbaI/BamHI (fragment 1) or KpnI/BamHI (fragment 2) and cloned in a single ligation step in pUC18ery digested with XbaI/KpnI resulting in pNZ4222. Plasmid pNZ4222 was transformed to L. lactis NZ9000 (pNZ4130) and single cross-over plasmid integrants were selected on plates containing erythromycin and tetracycline. One single Ery^{R}/Tet^{R} colony was obtained resulting from integration over the epsRA locus. This integrant was cultured in medium containing only tetracycline and Tet^{R} colonies were screened after 40 generations by replica plating on GM17 plates containing tetracycline or both erythromycin and tetracycline. A single colony was obtained that was erythromycin-sensitive (Ery^{S}) and that had lost the ropy phenotype. Deletion of the correct region was confirmed by PCR and Southern blotting and the plasmid was designated pNZ4200. In addition, a 0.9 kb fragment was amplified from pNZ4200 using primers EPSANCOI and EPSFR2 that was sequenced to confirm that the deletion was in-frame.

Polysaccharide production in L. lactis can be elevated by increasing the copy number of the plasmid encoding the polysaccharide biosynthesis genes (Boels et al., 2003). Therefore, the entire ΔepsABCD gene cluster from plasmid pNZ4200 including the eps promoter was cloned on the high copy vector pIL253. This was achieved by excising the 14 kb ΔepsABCD gene cluster from pNZ4200 as a NcoI fragment and subsequent cloning of the fragment in pIL253 carrying a NcoI site (Boels et al., 2003). The resulting plasmid was designated pNZ4220.

The 6.8 kb fragment encoding the cps14FGHIJKL genes were amplified from genomic DNA of S. pneumoniae serotype 14 in two separate PCR reactions. A 3.1 kb fragment encoding cps14JKL was amplified using primers cps14Jf and rev-cps14L and Pfx Platinum polymerase (Invitrogen) at the following PCR conditions: 15 s at 94° C., 30 s 46° C., 4.5 min at 68° C. A 3.9 kb amplicon encoding cps14FGHIJ was obtained using primers FWD-CPS14F and CPS14Jr and the PCR programme: 15 s at 94° C., 1 min at 40° C., 4.5 min at 68° C. After digestion with BamHI (site introduced in primers FWD-CPS14F and REV-CPS14L) and HindIII (site present in the cps14J gene), the two PCR fragments were subcloned in pNZ84 resulting in pNZ84cpsF-J and pNZ84cpsJ-L, respectively, and the inserts were verified by sequencing. The two fragments were re-isolated as BamHI/HindIII fragments and cloned in BamHI-digested pNZ84 in a single ligation reaction resulting in pNZ84cpsF-L. A 6.8 kb fragment encoding the cps14FGHIJKL genes was re-isolated from pNZ84cpsF-L by digestion with BamHI and introduced in L. lactis by cloning in similarly digested pNZ4220 yielding pNZ4230. The correct orientation of the cps14 genes was confirmed by both PCR and by digestion of pNZ4230.

The cps14BCDE genes of the cps14 gene cluster were cloned on a separate plasmid. Therefore, a 2.1 kb fragment containing the cps14CD genes and truncated cps14B (5′ end truncation) and cps14E (3′ end truncation) genes, was excised from pMK104 by digestion with SphI and XbaI and cloned in pNZ8020 resulting in pNZ4233. The 3′ end of cps14E was excised from pNZ4090 by digestion with. XbaI (sites present in the cps14E gene and in the multiple cloning site of pNZ4090) and cloned in pNZ4233. The resulting plasmid was designated pNZ4235. The cps14B gene was amplified from chromosomal S. pneumoniae DNA using primers CPS14Bf and CPS14Br. This PCR product was digested with EcoRI/KpnI and cloned in similarly digested pNZ8020 resulting in pNZ4231. The 5′ end of cps14B was introduced in pNZ4235 as a 0.6 kb fragment using the internal SacI restriction site present in the cps14B gene and the BamHI site from the multiple cloning site of pNZ4231 resulting in pNZ4237. For the construction of pNZ4238, a 2.2 kb PCR fragment encompassing cps14BCD was amplified from pNZ4237 using primers CPS14Deco and PEPS054f and Pwo polymerase. This amplicon was subsequently cloned in the SmaI site pNZ4090 carrying the truncated cps14E gene and the correct orientation of the insert was confirmed by PCR. Plasmid pNZ4221 was constructed via Ecl136II digestion and subsequent removal of a 415 bp epsD fragment from pNZ4206 and insertion of a cps14E PCR fragment (primers CPS14EF/CPS14ER) in the Ecl136II-digested plasmid.

TABLE 2
Oligonucleotides used in this study

Oligonucleotide sequence (5′→3′)

| | |
|---|---|
| EPSRF1 | ATTCTAGATTGAATCAAGTGGTAAATCTGC |
| EPSAR1 | CCATCTGGATCCATACTTTGACGCGAATAATTTTAAAAA |
| | TCCCTC |
| EPSFR2 | ATGGTACCTGACATAGTGTATTGGCTCG |
| EPSDF2 | GTCAAAGTATGGATCCAGATGGAGCTCTATTATCTCCAG |
| | TACC |
| EPSANcoI | CGGAATTCAAGGAGGCACTCACCATGGAGGAAACACAGG |
| | AACAG |
| CPS14Deco | GTTTGAATTCCTTTCCTAAGTTATTTTTTACC |
| PEPS054F | AAGTATTTCGCTATGTACACC |
| CPS14Bf | ACGGTACCGTAGTTAAAGCAGCTATACAGG |
| CPS14Br | CCGAATTCATAGTTGCAATACATCGATTTCC |
| FWD-CPS14F | CAATGGATCCGCGGCCGTAGTAAAATAACC |
| REV-CPS14L | AGCCGGATCCTCTCCATATCCTCTACCACC |
| CPS14Jf | CTAGATTCTGGGGAAGTATCC |
| CPS14Jr | ATTGATACCCTTTTACAGTCCC |
| CPS14EF | CGCTTCCTATGGAAATTATGG |
| CPS14ER | CGCTTCCTATGGAAATTATGG |

Underlined basepairs in oligonucleotide sequences indicate introduced restriction sites.

### Immunoblot Analyses

Serotype 14 polysaccharide production was analysed in cell pellets and supernatant of the L. lactis cultures by using immunodetection. Cells were grown in M17 medium (2% glucose) to an OD₆₀₀ of 0.15 and aliquoted in two cultures of which one was induced with 1 ng/ml nisin. Both induced and non-induced cultures were grown overnight and cells were pelleted by centrifugation. 10 μl of the supernatant was pipetted onto nitrocellulose filters. The cell pellets were washed once with phosphate-buffered saline (PBS) and resuspended to OD₆₀₀ =1 and 10 μl of the resuspended cells were pipetted onto nitrocellulose filters. Filters were blocked for 1 h at room temperature in 1% bovine serum albumin in PBS with 0.05% Tween 20 (PBS-T). For the detections of PS14, filters were incubated overnight at room temperature with 1:1000 dilution of antiserum against the capsular serotype 14 polysaccharide (Statens Serum Institute, Copenhagen, Denmark) as the primary antiserum. Filters were washed three times with PBS-T and then incubated with 1:2000 dilution of goat anti-rabbit immunoglobulin conjugated to horseradish peroxidase (Pierce, Rockford, USA). The filters were washed twice with PBS-T followed by one washing step using PBS. The conjugate was visualized using Supersignal substrate (Pierce) according to the instructions of the manufacturer.

For the analysis of tyrosine phosphorylated Cps14D and EpsB, cells were grown in M17 medium supplemented with 1% glucose. Cells were induced at an OD600=0.15-0.20 by the addition of nisinZ. Cells were harvested 3-4 hours after induction by centrifugation and the pellet was resuspended in 10 mM Tris [pH 8.0], 0.1 mM EDTA. Cells were mechanically disrupted in the presence of Zirconium glass beads (van der Meer et al., 1993). Cell debris was removed by centrifugation and the protein in the cell extract (CE) was boiled for 10 minutes in 2-times concentrated Laemmli buffer (Laemmli, 1970). Proteins were separated by SDS-PAGE (12.5% gel) and transferred onto nitrocellulose membranes by electroblotting (LKB 2051 Midget Multiblot). Membranes were blocked for one hour at room temperature using Tris-buffered saline (100 mM Tris [pH 7.4], 0.9% NaCl) with 0.05% Tween 20 (TBS-T)and 2% BSA. Monoclonal anti-phosphotyrosine antiserum (PT-66, Sigma) was used at a 1:1000 dilution in TBS with 0.05% Tween 20 and 0.5% BSA and incubated overnight. Membranes were washed as described above and incubated with 1:2,000 dilution of goat anti-mouse immunoglobulin conjugated to horseradish peroxidase (Pierce). Binding of the secondary antibody was visualized using chloronaphtol and H₂O₂.

### Isolation and Analysis of Polysaccharide

Cells were grown in CDM containing 2% glucose to an OD600=0.15-0.20. Cultures were subsequently divided over two tubes and one tube was induced by the addition of 1 ng/ml nisin while the other tube was not induced. Both induced and non-induced cells were incubated overnight. Polysaccharide was isolated from the cultures as described by Looijesteijn and Hugenholtz (1999). For the type 14 polysaccharide producing strain, both the above described method and the protocol described by Karlsson et al. (1998) for isolation of capsular polysaccharides were used.

### NMR Analysis

Polysaccharide was isolated from 1 L culture of L. lactis NZ9000 harbouring pNZ4206 and pNZ4230. Cells were induced as described above and polysaccharide was harvested from the supernatant by centrifugation (10 min, 15,000×g). The supernatant was adjusted to pH 7 by adding 10 M NaOH and concentrated by ultrafiltration (MWCO 20,000 Da). The concentrated polysaccharide was dialysed against running tap water, and protein was removed by the addition of 600 μg proteinase K in 40 mM Tris [pH 8.0], 10 mM MgCl₂ and 10 mM CaCl₂ and an overnight-incubation step at 55° C. The proteinase-treated polysaccharide solution was dialyzed overnight against running tap water, lyophilized and dissolved in 0.1 M NaNO₃. The solution was fractionated by size-exclusion chromatography (SEC) using TSK-gel 6000 PW columns (Phenomenex) and 0.1 M NaNO₃ as the eluent. The eluent from the column was analyzed on-line by both refractive index (RI) and UV detection at 280 nm. EPS-containing fractions were collected, dialyzed against Millipore water and lyophilized. Lyophilized samples were dissolved in 99.9 atom % D₂O and NMR spectra were taken at 400 MHz of the type 14 polysaccharide produced in L. lactis and of type 14 polysaccharide purified from S. pneumoniae (American Type Culture Collection, Manassas, USA).

### Results

To illustrate the current invention, CPS from Streptococcus pneumoniae serotype 14, for which the complete gene cluster that directs its biosynthesis is known (Kolkman et al., 1997), was produced in a non-pathogenic, non-invasive Gram-positive host cell, such as in this example Lactococcus lactis.

The type 14 polysaccharide consists of a linear backbone of →6)-β-D-GIcpNAc-(1→3)-β-D-Ga1p-(1→4)-β-D-G1cp-(1→repeating units with a β(1→4)-Ga1p residue linked to C4 of each N-acetylglucosamine residue. The complete genes cluster encompasses 12 genes (cps14A to cps14L) which are transcribed as a single transcriptional unit (Kolkman et al., 1997). The cps 14 gene cluster is organized in the typical cassette-like structure with the common region flanked by the type-specific genes including the polymerase and repeat unit transporter.

Complex pneumococcal CPS production in L. lactis according to the current invention is accomplished by cloning the three common lactococcal genes (epsABC) plus the glycolsyltransferase-encoding gene epsD on one plasmid, and the remaining type specific genes on a second plasmid. Interestingly, only the expression of the type 14-specific genes in combination with the eps B40 common genes resulted in type 14 polysaccharide production.

Cloning of the Expression System for Pneumococcal Type 14 Polysaccharide in L. lactis

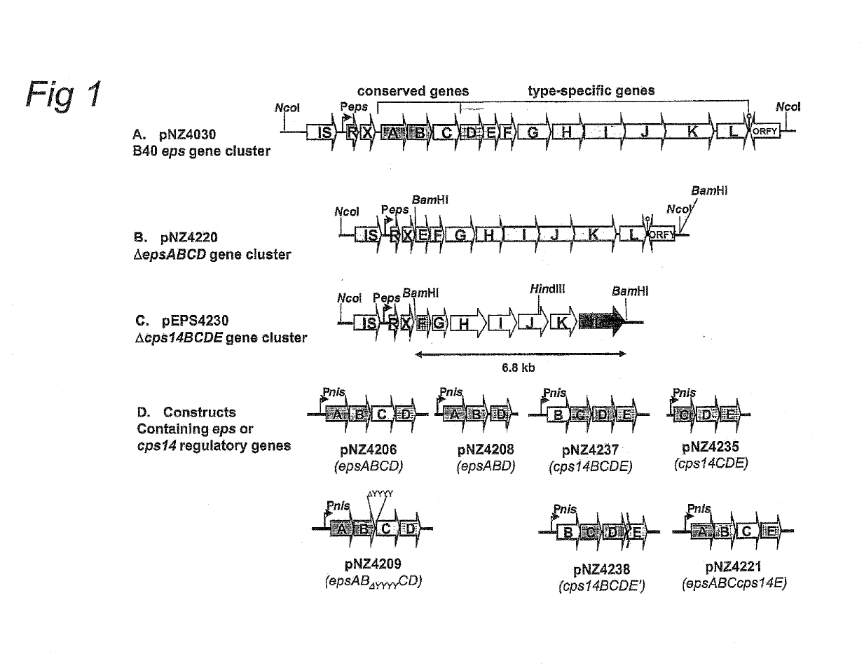

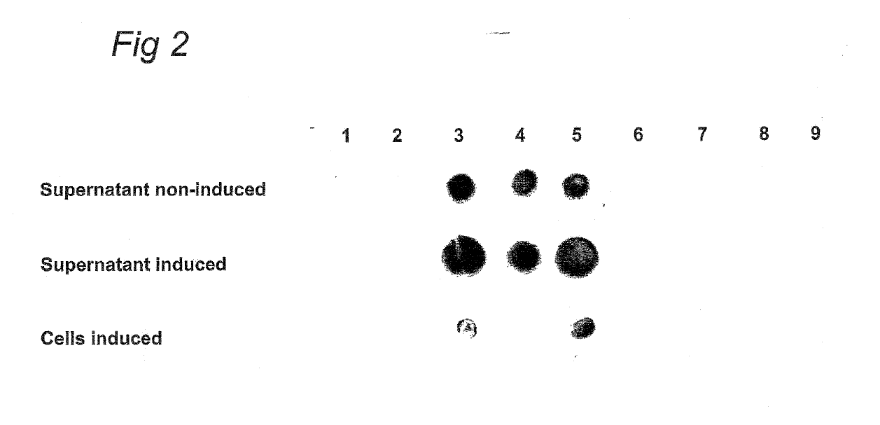

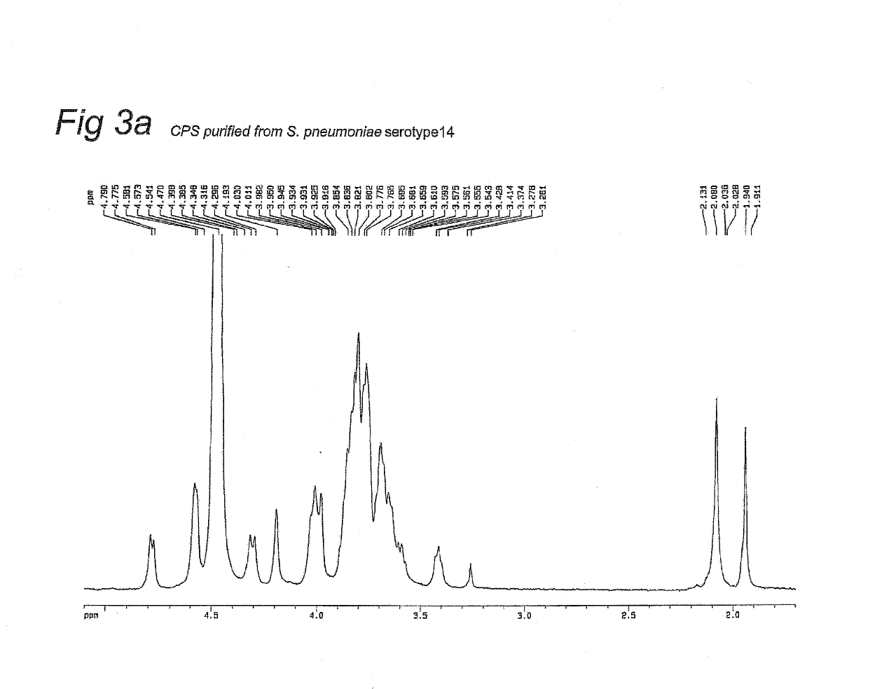

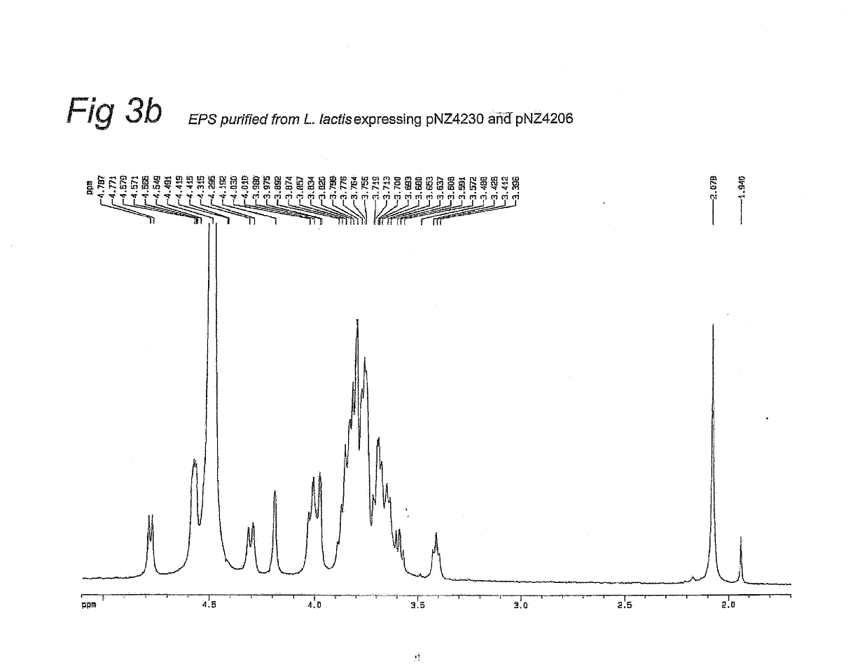

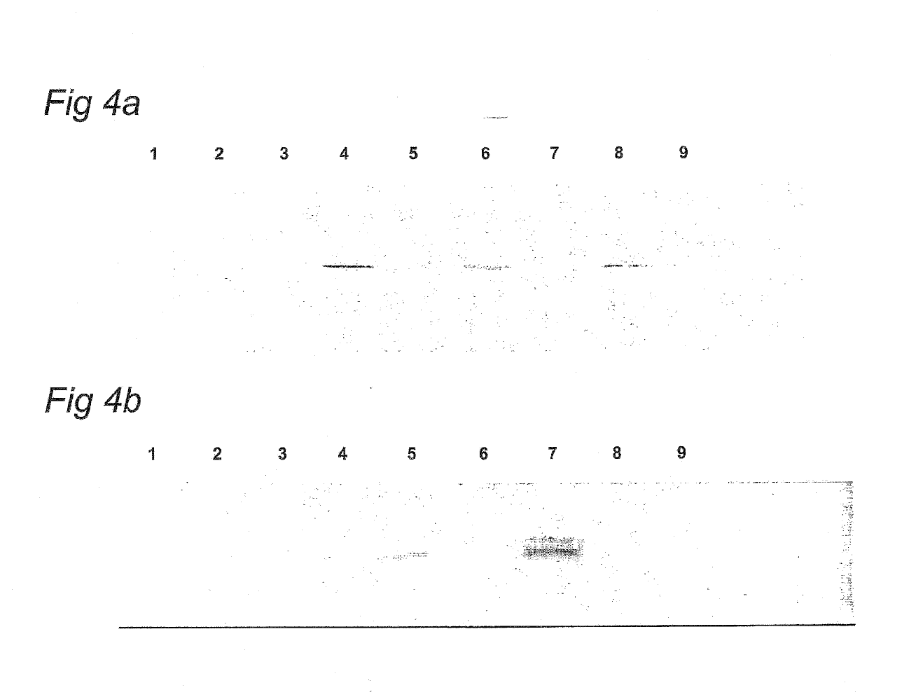

For the synthesis of type 14 polysaccharides (polysaccharide), the sugar nucleotides UDP-Gal, UDP-GlcNAc, and UDP-Glc are used as building blocks (Kolkman et al., 1997). These activated sugars are formed in L. lactis from intermediates of the central carbon metabolism (www.kegg.genome.ad.jp). For the expression of type 14 polysaccharide in L. lactis, we modified the expression system that was already proven successful in L. lactis for both homologous and heterologous expression of eps genes in our laboratory (Nierop Groot et al., 2004). FIG. 1 shows a schematic overview of the plasmids used for lactococcal and pneumococcal polysaccharide production in L. lactis. In this expression system, the conserved cassette-like organization of polysaccharide biosynthesis gene clusters in bacteria is used. The cps14 gene cluster is divided over two separate plasmids. The cps14 type-specific genes, encoding glycosyltransferases and the polymerase and export proteins, are cloned on the pNZ4220 derived plasmid. Therefore, the epsEFGHIJKL or fY genes from the lactococcal eps gene cluster were excised from plasmid pNZ4220 using the flanking BamHI restriction sites leaving the eps promoter sequence and epsRX. A 6.8 kb fragment was excised from plasmid pNZ84F-L (Material and Methods) by digestion with BamHI and ligated in the similarly digested pNZ4220 resulting in plasmid pNZ4230. In pNZ4230, expression of the cps14 type-specific genes is under control of the constitutive promoter of the lactococcal eps gene cluster. The conserved regions, encoding the regulatory genes, and the priming glycosyltransferase, of both the lactococcal eps and the pneumococcal cps14 gene clusters were cloned under control of the nisin-inducible promoter resulting in pNZ4206 (epsABCD) and pNZ4237 (cps14BCDE). Hence, cps14A, that is expected to be involved in transcriptional activation of the cps gene cluster (Cieslewicz et al. (2001), is not present in this system. Expression of the cps14 genes in this system is driven by the constitutive B40 eps promoter and by the nisin-inducible promoter. It was previously shown that introduction of the cps14E gene in an epsD mutant of L. lactis can restore EPS production (Van Kranenburg et al., 1999) since the glycosyltransferases EpsD and Cps14E both have specificity for glucose (van Kranenburg et al., 1999; Kolkman et al., 1997). Plasmids pNZ4206 and pNZ4237 were transformed to L. lactis harbouring pNZ4230.

Immunodetection of Type 14 Polysaccharide Production in L. lactis

Induced and non-induced cultures of L. lactis harbouring pNZ4230 in combination with either pNZ4206 (epsABCD)or pNZ4237 (cps14BCDE) were tested for type 14 polysaccharide production using immunodetection. In addition, the constructs PNZ4209 (epsAB_{ΔYYYY}CD), pNZ4208 (epsABD), PNZ4235 (cps14CDE), pNZ4238 (cps14BCDE′) and pNZ4221 (epsABC+cps14E) were tested in combination with pNZ4230.

As negative controls, strain NZ9000 harbouring pNZ4230, and NZ9000 harbouring pNZ4220 in combination with pNZ4206 were used. Both nisin-induced and non-induced cells were grown overnight in M17 medium supplemented with 2% glucose. Cells were centrifuged and both supernatant and cells were spotted onto nitrocellulose membranes and the presence of polysaccharide was detected using type 14-specific antiserum. A strong signal was detected in the supernatant of the induced L. lactis strain harbouring pNZ4230 in combination with pNZ4206, pNZ4208 and pNZ4209 and to a lesser extend in the non-induced cells. This signal in the non-induced cells results from leakage of the nisA promoter since no signal was detected for the negative control strain in lane 2 that harbors only pNZ4230. No signal was detected in the B40-EPS producing strain in lane 1 showing that the serum specifically detects type 14 polysaccharide. Surprisingly, no signal was detected for the L. lactis strain harbouring pNZ4230 in combination with pNZ4237 (lane 7). This was unexpected since plasmid pNZ4237 contains the regulatory genes from the pneumococcal cps 14 gene cluster. Construct pNZ4208 in lane 5 lacks the epsC gene but L. lactis harbouring this construct produce type 14 polysaccharide. This indicates that in L. lactis polysaccharide production is not strictly dependent on the presence of a functional epsC gene. Deletion of 7 amino acids at the C-term of EpsB (construct pNZ4209 in lane 4) appears to reduce the amount of polysaccharide produced. Interestingly, type 14 polysaccharide was mainly released in the culture supernatant. A weak signal was detected in the cell suspension of L. lactis harbouring pNZ4230/pNZ4206 that was most likely loosely associated polysaccharide that was not completely removed during the washing step. Plasmid pNZ4237 contains the complete cps14E gene whereas previous reports show that an additional ribosomal binding site is present in cps14E resulting in a truncated, but active, glycosyltransferase missing the first 98 amino acids (Kolkman et al., 1997). It has been suggested that the N-terminal half of the priming glycosyltransferases may be involved in the release of the undecaprenyl-linked repeat unit (Wang et al., 1996). The lactococcal homolog, EpsD, lacks this domain and contains only the conserved domains necessary for the glucosyltransferase activity. To test whether this additional domain in Cps14E was interfering with polysaccharide production in L. lactis, the intact cps14E gene in pNZ4237 was replaced by the truncated cps14E resulting in pNZ4238. However, no signal was detected for the strain harbouring pNZ4230 in combination with pNZ4238. Replacement of epsD in construct pNZ4206 for the cps14E gene also prevented type 14 polysaccharide production. Data by Van Kranenburg et al. (1999) and in Table 3 show that the glucosyltransferase Cps14E is functional in B40 polysaccharide biosynthesis in L. lactis. This indicates that type 14 polysaccharide biosynthesis in L. lactis requires the presence of EpsA, EpsB and EpsD and that EpsC is optionally.

TABLE 3
Polysaccharide (PS) isolated from the culture
supernatant of L. lactis strains.

| Strain NZ9000 | | | | Polymer |
|---|---|---|---|---|
| harbouring | unin- | PS (mg/l)^{a} | PS | type |
| plasmids | duced | induced^{b} | (mg/L · OD₆₀₀)^{c} | produced^{d} |
| pNZ4230 + pNZ4237 | <1 | <1 | — | |
| pNZ4230 + pNZ4235 | <1 | <1 | — | |
| pNZ4230 + pNZ4206 | <1 | 25 | 12 | type 14 |
| pNZ4230 + pNZ4209 | <1 | 12 | 6 | type 14 |
| pNZ4230 + pNZ4208 | <1 | 21 | 11 | type 14 |
| pNZ4230 + pNZ4221 | <1 | <1 | — | |
| pNZ4220 + pNZ4237 | <1 | 31 | 13 | B40 |
| pNZ4220 + pNZ4235 | <1 | 6 | 3 | B40 |
| pNZ4220 + pNZ4206 | <1 | 110 | 56 | B40 |
| pNZ4220 + pNZ4238 | <1 | 37 | 15 | B40 |
| pNZ4220 + pNZ4221 | <1 | 92 | 23 | |

^{a}The values presented are averages of at least two independent experiments and varied from the mean by no more than 7%.
^{b}Cells were induced with 1 ng/ml nisin as described in Materials and Methods
^{c}Polysaccharide production corrected for the final optical density (OD₆₀₀)
^{d}B40 refers to the lactococcal B40 polysaccharide; type 14: polysaccharide reacting with pneumococcal serotype 14 specific antiserum.

Isolation of Polysaccharide from L. lactis Culture

The polysaccharide produced by the L. lactis strains were analyzed by size-exclusion chromatography followed by multi-angle light scattering (SEC-MALLS). L. lactis harbouring pNZ4230 and pNZ4206 produced 25 mg of the type 14 polysaccharide per liter (Table 3). This is 23% of the amount of B40 polysaccharide produced by L. lactis harbouring pNZ4220 and pNZ4206. Polysaccharide was also produced in the strains harbouring pNZ4208 and pNZ4209 although production in the latter strain was significantly lower. Either no polysaccharide or amounts below the detection limit of 1 mg/L were produced in non-induced cells. Immunodetection is a more sensitive detection method and explains the signal obtained for non-induced cells of L. lactis (pNZ4230, pNZ4206) in FIG. 2. As already suggested by the immunodetection experiment, no polysaccharide could be isolated from L. lactis harbouring pNZ4230 in combination with pNZ4237. A different method, developed for isolation of pneumococcal capsular polysaccharides (Karlsson et al., 1998), was used in addition and confirmed that no polysaccharide was produced. Interestingly, L. lactis harbouring pNZ4220 in combination with pNZ4237 or pNZ4238 produced 31 mg/L or 37 mg/L of B40 polysaccharide, respectively. This confirms that the cps14BCDE genes are functional in L. lactis and that polysaccharide production is comparable for the intact and the truncated cps14E constructs. Table 3 further shows that cps14E is functional in combination with the epsABC genes in the B40 polysaccharide producing strain (pNZ4220+pNZ4221) but not in the strain harbouring pNZ4230. Pneumococcal type 14 polysaccharide is thus only produced in L. lactis under control of the epsABCD genes (either in the presence or absence of epsC).

### Immunodetection of Phosphorylated Tyrosine Residues

Reversible phosphorylation of tyrosine residues present in CpsD are known to regulate capsule production in S. pneumoniae (Bender and Yother, 2001; Bender et al., 2003). FIG. 4 shows that Cps14D is phosphorylated in the strains harbouring pNZ4230 in combination with the cps14BCDE genes (Lane 4), the cps14CDE genes (Lane 6), the cps14BCDE′ genes (Lane 8) and in the epsABCcps14E construct (Lane 9). Interestingly, these strains did not produce type 14 polysaccharide. In L. lactis harbouring pNZ4220, phosphorylated tyrosine was only detected when polysaccharide production was under control of Cps14BCDE or Cps14CDE and the polysaccharide production was largely reduced compared to the EpsABCD regulatory proteins. This indicates that tyrosine phosphorylation negatively effects polysaccharide biosynthesis in L. lactis. The EpsB protein was phosphorylated in L. lactis harbouring pNZ4221 (epsABCcps14E) in combination with pNZ4230 but not in combination with pNZ4200. This indicates that the pneumococcal priming glucosyltransferase can block polysaccharide biosynthesis and thus is preferably exchanged for the lactococcal glucosyltransferase.

### NMR Analysis

Because the chemical structure of the L. lactis produced polysaccharide is identical to the immunogenic S. pneumoniae polysaccharide, it is anticipated without doubt that the L. lactis produced polysaccharide will evoke a protective immune response. This is further supported by data from Gilbert et al (2000) that show immune responses in mice elicited by L. lactis produced type 3 polysaccharide is identical to those observed for the S. pneumoniae produced polysaccharide.

The proton NMR spectra of the type 14 polysaccharide produced in L. lactis and the type 14 polysaccharide produced in S. pneumoniae were taken and show identical spectra (FIG. 3). A small impurity present in the CPS isolated from S. pneumoniae serotype 14 resulted in an additional peak at 3.27 ppm not present in the lactococcal isolate. However, the spectra clearly show that the structure of the polysaccharide produced in L. lactis is identical to the native serotype 14 polysaccharide produced in S. pneumoniae.

Summarizing, the example demonstrates for the first time heterologous expression and production of complex type Gram-positive polysaccharide from a pneumococ in a non-pathogenic and/or non-invasive Gram-positive host cells.

The production level as achieved by Gilbert et al., 2000, WO 98/31786 for type 3 pneumococcal polysaccharide production in L. lactis was 120 mg/L. This is a simple type polysaccharide. Complex type 14 pneumococcal polysaccharide is produced at 25 mg/l in this example, and may be further enhanced and optimized by culturing the host bacteria under various conditions. Type 14 polysaccharide is more complex and most other pneumococcal polysaccharides are synthesized via highly similar mechanisms. Improvements in production levels may be achieved via an increase of UDP-GlcNAc levels in L. lactis, as UDP-glucose and UDP-galactose are most likely not limiting, according to (Boels et al., 2003). The example also illustrates another advantage of the current invention; type 14 polysaccharide produced in S. pneumoniae is produced as a capsule, in L. lactis secreted in the medium. The method for heterologous production of complex polysaccharide according to this invention provides advantages in terms of allowing safe and convenient, production of polysaccharide from non-pathogenic Gram-positive bacteria, but also allows a convenient isolation from the culture medium in which the heterologously produced polysaccharide types are secreted and less protein contamination.

### REFERENCES

Arrecubieta, C., R. Lopez, and E. Garcia. 1996. Type 3-specific synthase of Streptococcus pneumoniae (Cap3B) directs type 3 polysaccharide biosynthesis in Escherichia coli and in pneumococcal strains of different serotypes. J. Exp. Med. 184: 449-455.

Aravind, L. and E. V. Koonin. 1998. Phosphoesterase domains associated with DNA polymerases of diverse origins. Nucleic Acids Res. 15;26(16):3746-52.

Boels, I. C., R. van Kranenburg, M. W. Kanning, B. Pong Chong, W. M. de Vos, and M. Kleerebezem. 2003. Increased exopolysaccharide production in Lactococcus lactis due to increased levels of expression of the NIZO B40 eps gene cluster. Appl. Environ. Microbiol. 69: 5029-5031.

Boels, I. C., A. Ramos, M. Kleerebezem, and W. M. de Vos. 2001. Functional analysis of the Lactococcus lactis galU and galE genes and their impact on sugar nucleotide and exopolysaccharide biosynthesis. Appl. Environm. Microbiol. 67: 3033-3040.

Bender, M. H., R. T. Cartee, and J. Yother. 2003. Positive correlation between tyrosine phosphorylation of CpsD and capsular polysaccharide production in Streptococcus penumoniae. J. Bacteriol. 185: 6057-6066.

Bender, M. H., and Y. Yother. 2001. CpsB is a modulator of capsule-associated tyrosine kinase activity in Streptococcus penumoniae. J. Biol. Chem. 276: 47966-47974.

Campbell, J. A., G. J. Davies, V. Bulone, and B. Henrissat. 1997. A classification of nucleotide-diphospho-sugar glycosyltransferases based on amino acid sequence similarities. Biochem J. 15: 929-39.

Cartee, R. T., W. Thomas Forsee, J. S. Schutzbach, and Y. Yother. 2000. Mechanism of type 3 capsular polysaccharide synthesis in Streptococcus pneumoniae. J. Biol. Chem. 275: 3907-3914.

Cieslewicz, M. J., D. L. Kasper, Y. Wang, and M. R. Wessels. 2001. Functional analysis in type Ia group B Streptococcus of a cluster of genes involved in extracellular polysaccharide production by diverse species of streptococci. J. Biol. Chem. 276: 139-146.

Conçalves, V. M. M., M. Takagi, R, B. Lima, H. Massaldi, R. C. Giordano, and M. M. Tanizaki. 2003. Purification of capsular polysaccharide from Streptococcus pneumoniae serotype 23F by a procedure suitable for scale-up. Biotechnol. Appl. Biochem. 37: 283-287.

De Ruyter, P. G. G. A., O. P. Kuipers, M. M. Beerthuyzen, I. J. Van Alen-Boerrigter, and W. M. De Vos. 1996. Functional analysis of promoters in the nisin gene cluster of Lactococcus lactis. J. Bacteriol. 178:3434-3439.

De Vos, W. M. P., H. de Haard, and I. Boerrigter. 1989. Cloning and expression of the Lactococcus lactis subsp. cremoris SK11 gene encoding an extracellular serine proteinase. Gene 85: 169-176.

Fath, M. J., and R. Kolter. 1993. ABC transporters: bacterial exporters. Microbiol. Rev. 57: 995-1017.

Gilbert, C., Robinson, K., Le Page, R. W. F., and J. M. Wells. 2000. Heterologous expression of an immunogenic pneumococcal type 3 polysaccharide in Lactococcus lactis. Infection and Immunity 68: 3251-3260.

Guidolin, A., J. K. Morona, R. Morona, D. Hansman, and J. C. Paton. 1994. Nucleotide sequence analysis of genes essential for capsular polysaccharide biosynthesis in Streptococcus pneumoniae type 19F. Infect, Immun. 62: 5384-5396.

Jolly, L., J. Newell, I. Porcelli, S. J. F. Vincent, and F. Stingele. 2002. Lactobacillus helveticus glycosyltransferases: from genes to carbohydrate synthesis. Glycobiology 12: 319-327.

Jolly, L., and F. Stingele. 2001. Molecular organization and functionality of exopolysaccharide gene clusters in lactic acid bacteria. Int. Dairy J. 11: 733-745.

Henrichsen, J. 1999. Typing of Streptococcus pneumoniae: past present and future. Am. J. Med. 26: 50S-54S.

Karlsson, C., P. E. Jansson, and U. B. Sørensen. 1998. The chemical structures of the capsular polysaccharides from Streptococcus pneumoniae types 32F and 32A. Eur. J. Biochem. 255: 296-302.

Kleerebezem, M., Boekhorst, J. & 17 other authors. 2003. Complete genome sequence of Lactobacillus plantarum WCFS1. PNAS 100, 1990-1995.

Kolkman, M. A. B., B. A. M. van der Zeijst, and P. J. M. Nuijten. 1997. Functional analysis of glycosyltransferases encoded by the capsular polysaccharide biosynthesis locus of Streptococcus pneumoniae serotype 14. J. Biol. Chem. 272: 19502-19508.

Kolkman, M. A. B., Wakarchuk, W., Nuijten, P. J. M., and B. A. M. van der Zeijst. 1997. Capsular polysaccharide synthesis in Streptococcus pneumoniae serotype 14: molecular analysis of the complete cps locus and identification of genes encoding glycosyltransferases required for the biosynthesis of the tetrasaccharide subunit, Mol. Microbiol. 26: 197-208.

Laemmli, U. K. 1970. Cleavage of the structural proteins during the assembly of the head of bacteriophage T4. nature (London) 227: 680-685.

Lamothe, G. T., L. Jolly, B. Mollet, F. Stingele. 2002. Genetic and biochemical characterization of exopolysaccharide biosynthesis by Lactobacillus delbrueckii subsp. bulgaricus. Arch. Microbiol. 178: 218-228.

Looijesteijn, P. J., and J. Hugenholtz. 1999. Uncoupling of growth and exopolysaccharide production by Lactococcus lactis subsp. cremoris NIZO B40 and optimisation of its synthesis. J. Biosci. And Bioeng. 88: 178-182.

Llull, D., E. Garcia, and R. Lopez. 2001. Tts, a processive β-glucosyltransferase of Streptococcus pneumonia, directs the synthesis of the branched type 37 capsular polysaccharide in pneumococcus and other Gram-positive species. J. Biol. Chem. 276: 21053-21060.

Morona, J. K., R. Morona, and J. C. Paton. 1997. Molecular and genetic characterization of the capsule biosynthesis locus of Streptococcus pneumoniae type 19B. J. Bacteriol. 179: 4953-4958.

Morona, J. K., R. Morona, D. C. Miller, and J. C. Paton. 2003. Mutational analysis of the carboxy-terminal (YGX)₄ repeat domain of CpsD, an autophosphorylating tyrosine kinase required for capsule biosynthesis in Streptococcus pneumoniae. J. Bacteriol. 185: 3009-3019.

Nierop Groot, M. N., and M. Kleerebezem. 2004. Manuscript in preparation.

Platteeuw, C., G. Simons, and W. M. de Vos. 1993. Use of the Escherichia coli (3-glucoronidase (gusA) gene as a reporter gene for analyzing promoters in lactic acid bacteria. Appl. Environ. Microbiol. 60: 587-593.

Sambrook, J., Fritsch, E. F., & Maniatis, T. (1989). Molecular cloning: a laboratory manual, 2^{nd} ed. Cold Spring Harbor Laboratory Press, Cold Spring Harbor, N.Y.

Sørensen, U. B. S., Henrichsen, J., Chen, H-C., and S. C. Szu. 1990. Covalent linkage between the capsular polysaccharide and the cell wall peptidoglycan of Streptococcus pneumoniae revealed by immunochemical methods. Microbial Pathogenesis 8:325-334.

Stingele, F., J-R. Neeser, and B. Mollet. 1996. Identification and characterization of the eps (exopolysaccharide) gene cluster from Streptococcus thermophilus Sfi6. J. bacteriol. 178: 1680-1690.

Stingele, F., Vincent, S. J., Faber, E. J., Newell., J. W., Kamerling, J. P., and J. R. Neeser. 1999. Introduction of the exopolysaccharide gene cluster from Streptococcus thermophilus Sfi6 into Lactococcus lactis MG1363: production and characterization of an altered polysaccharide. Mol. Microbiol. 32: 1287-1295.

Van der Meer, J. R., J. Polman, M. M. Beerthuyzen, R. J. Siezen, O. P. Kuipers, and W. M. de Vos. 1993. Characterization of the Lactococcus lactis nisinA operon genes nisP, encoding a subtilisin-like protease involved in precursor processing, and nisR, encoding a regulatory protein involved in nisin biosynthesis. J. Bacteriol. 175: 2578-2588.

Van Kranenburg, R., J. D. Marugg, I. V. Swam, N. J. Norwin, J. Willem, and W. M. de Vos. 1997. Molecular characterisation of the plasmid-encoded eps gene cluster essential for exopolysaccharide biosynthesis in Lactococcus lactis. Mol. Microbiol. 24: 387-397.

Van kranenburg, R., H. R. Vos, I. Van Swam, M. Kleerebezem, and W. M. de Vos. 1999. Functional analysis of glycosyltransferase genes from Lactococcus lactis and other gram-positive cooci: complementation, expression and diversity. J. Bacteriol. 181: 6347-6353.

Velasco et al., 1995. Streptococcus pneumoniae: virulence factors, pathogenesis and vaccines. Microbiol. Rev. p. 591-603.

Wang, L., D. Liu, and P. R. Reeves. 1996. C-terminal half of Salmonella entericaWbaP (RfbP) is the galactosyl-1-transferase domain catalyzing the first step of O-antigen synthesis. J. Bacteriol. 178: 2598-2604.

Weintraub, A. 2003. Immunology of bacterial polysaccharides. Review. Carbohydrate Research 338: 2539-2547.

Whatmore, A. M., A. Efstratiou, A. P. Pickerill, K. Broughton, G. Woodward, D. Sturgeon, R. George, and C. G. Dowson. 2000. Genetic relationships between clinical isolates of Streptococcus pneumoniae, Streptococcus oralis, and Streptococcus mitis: characterization of “a typical” pneumococci and organisms allied to S. mitis harbouring S. pneumoniae virulence factor-encoding genes. Infect. Immun. 68: 1374-1382.

Whitfiel. 1995. Biosynthesis of lipopolysaccharide O-antigens. Trends Microbiol 3: 178-185.

1. A non-pathogenic, non-invasive Gram-positive bacterium that comprises:
a) a first heterologous DNA fragment comprising capsular polysaccharide (CPS) serotype specific genes of a Gram-positive bacterial species;
b) a second DNA fragment comprising the common, regulatory genes and a priming-glycosyltransferase obtained from a Gram-positive bacterium different from the bacterium under a); and,
c) and upon expression of said fragments produces heterologous polysaccharides of the bacterial species under a).

2. The bacterium according to claim 1 wherein the serotype specific cps genes of the pathogenic and/or invasive Gram-positive bacterial species are from a species producing complex type CPS, wherein the CPS comprises a polymer of repetitive oligosaccharide units that are synthesized via lipid-linked intermediates.

3. The bacterium according to claim 1 wherein the polysaccharides produced under c) are secreted into the extracellular space.

4. The bacterium according to claim 1 wherein the bacterium is selected from the group of non-pathogenic, non-invasive, Gram-positive bacteria consisting of species from the genera Lactobacillus, Lactococcus, Pediococcus, Carnobacterium, Bifidobacterium, and Oenococcus, and of the species Bacillus subtilis, Streptococcus thermophilus.

5. The bacterium according to claim 1 wherein the serotype specific sequences are obtained from the group of pathogenic and/or invasive Gram-positive complex CPS producing bacteria consisting of Streptococcus pneumoniae, Enterococcus faecalis, Streptococcus mutans, Streptococcus pyogenes, Streptococcus agalactiae, S. epidermidis, Streptococcus gordonii, Streptococcus mitis, Streptococcus oralis, Streptococcus equi, Bacillus anthracis and Staphylococcus aureus.

6. The bacterium according to claim 5 wherein the serotype specific genes are from a Streptococcus pneumoniae strain producing complex capsular polysaccharides serotype 1, 2, 4, 5, 6A, 6B, 7A, 7B, 7C, 7F, 8, 9A, 9L, 9N, 9V, 10A, 10B, 10C, 11A, 11B, 11C, 11F, 12B, 12F, 13, 14, 15E, 15A, 15B, 15C, 16F, 16A, 17F, 17A, 18A, 18B, 18C, 19F, 19A, 19B, 19C, 20, 21, 22F, 22A, 23A, 23B, 24F, 24A, 24B, 25F, 25A, 27, 28F, 28A, 29, 33F, 33A, 33B, 33C, 10F, 11D, 12A, 18F, 23F, 31, 32F, 32A, 33D, 34, 35F, 35A, 35B, 35C, 36, 38, 39, 40, 41F, 41A, 42, 43, 44, 45, 46, 47F, 47A and 48.

7. The bacterium according to claim 1 wherein said expressed common or regulatory genes and the priming glycosyltransferase comprise at least the common, regulatory genes epsA or cpsC, epsB or epsD, and epsD or cpsE and optionally epsC or cpsB.

8. The bacterium according to claim 7 wherein the epsA encoded protein shares at least 20% amino acid identity with Lactococcus lactis EpsA, wherein the epsB encoded protein shares at least 20% amino acid identity with Lactococcus lactis epsB and wherein the epsD encoded protein shares at least 30% amino acid identity with Lactococcus lactis EpsD.

9. A DNA vector comprising a DNA fragment encoding Gram-positive complex CPS serotype specific cps genes, wherein the serotype specific genes are selected from the group consisting of the serotype specific genes present in a capsular polysaccharide gene (CPS) cluster.

10. The vector according to claim 9 wherein one or more serotype specific genes are selected from the group of serotype specific cps genes consisting of cpsE, cpsF, cpsG, cpsH, cpsI, cpsJ, cpsK, cpsL or homologs thereof.

11. The vector according to claim 10 wherein the serotype specific cps genes are obtained from the group of CPS producing Streptococcus pneumoniae strains, that produce a complex CPS of serotype 1, 2, 4, 5, 6A, 6B, 7A, 7B, 7C, 7F, 8, 9A, 9L, 9N, 9V, 10A, 10B, 10C, 11A, 11B, 11C, 11F, 12B, 12F, 13, 14, 15F, 15A, 15B, 15C, 16F, 16A, 17F, 17A, 18A, 18B, 18C, 19F, 19A, 19B, 19C, 20, 21, 22F, 22A, 23A, 23B, 24F, 24A, 24B, 25F, 25A, 27, 28F, 28A, 29, 33F, 33A, 33B, 33C, 10F, 11D, 12A, 18F, 23F, 31, 32F, 32A, 33D, 34, 35F, 35A, 35B, 35C, 36, 38, 39, 40, 41F, 41A, 42, 43, 44, 45, 46, 47F, 47A and 48.

12. The DNA vector according to any one of claims 9 to 11 wherein the serotype specific cps genes are under transcriptional control of an EPS or CPS gene cluster regulatory sequences from a Gram-positive bacterium, said bacterium being a different species from the Gram-positive bacterium of which the serotype specific genes were obtained.

13. The DNA vector according to claim 12 wherein the serotype specific genes are comprised within a polycistronic transcriptional unit.

14. The vector according to claim 12 wherein the vector does not comprise one or more of the functional common regulatory eps genes epsA or cpsC, epsB or cpsD, epsD or cpsD and optionally epsC or cpsB.

15. A bacterium according to claim 1, wherein the bacterium comprises a vector according to any one of claims 9 to 14.

16. Method for the heterologous production of complex capsular polysaccharides (CPS) in a non-pathogenic, non-invasive Gram-positive bacterium, comprising the steps of;
a) culturing the bacterium according to any of claims 1 to 8, under conditions conducive of CPS production,
b) and optionally, recovery of the produced complex CPS.

17. The method according to claim 16 wherein the bacterial cells and the culture medium are separated and the CPS is recovered in the culture medium, or optionally isolated from the culture medium.

18. A pharmaceutically acceptable composition comprising the bacterium according to any one of claims 1 to 7, and at least one excipient or immunogenic adjuvant.

19. A pharmaceutically acceptable composition comprising complex CPS obtained from a non-pathogenic, non-invasive Gram-positive bacterium according to any one of claims 1 to 6, and at least one excipient or immunogenic adjuvant.

20. The composition according to claim 19 wherein the complex CPS is physically linked to an immunogenic molecule.

21. The composition according to claim 19 wherein the immunogenic molecule is selected from the group of immunogenic proteins consisting of: tetanus toxoid, diphtheria toxoid, meningococcal outer membrane proteins and diphtheria protein CRM₁₉₇.
